(19) United States
(12) Patent Application Publication
(10) Pub. No.: US 2010/0292280 A1
(43) Pub. Date: Nov. 18, 2010

(54) ANTI-PYRETIC VASODILATORS

(76) Inventor: Oron Zachar, Tel Aviv (IL)

Correspondence Address: Daniel Feigelson, Lev Hamada Building, Second Floor, 7 Oppenheimer St., Rehovot, IL

(21) Appl. No.: 12/595,237

(22) PCT Filed: Apr. 14, 2008
(86) PCT No.: PCT/IL08/00508

§ 371 Date: Oct. 8, 2009

Related U.S. Application Data
(60) Provisional application No. 60/911,894, filed on Apr. 15, 2007.
(60) Provisional application No. 60/911,900, filed on Apr. 15, 2007.
(60) Provisional application No. 60/914,369, filed on Apr. 27, 2007.
(60) Provisional application No. 60/946,157, filed on Jun. 26, 2007.
(60) Provisional application No. 60/980,849, filed on Oct. 18, 2007.
(60) Provisional application No. 61/012,934, filed on Dec. 12, 2007.
(60) Provisional application No. 61/013,484, filed on Dec. 13, 2007.

(51) Int. Cl. A61K 31/455; A61P 29/00

(52) U.S. Cl. 514/356

(57) ABSTRACT

The invention provides vasodilating medication as means for lowering fever when administered to humans in need of such treatment. In particular, the use of B3 vitamin substances and Nitric Oxide-donor ingredients in compositions intended for use in reducing fever is introduced. The core composition substances can be used effectively on their own. Yet, in combination with anti-pyretic substances such as Aspirin, Acetaminophen, and Ibuprofen, the present invention enables the use of reduced dosage of composing substances for achievement of desired fever reduction effect. In addition, an optional addition of sweat inducing plant extracts in any of the noted compositions leads to a synergistic effect of reducing fever by increase of both skin blood flow and perspiration.

### FIELD OF THE INVENTION

The invention relates generally to the field of anti-pyretic treatment. More specifically, the present invention relates to methods of reducing fever employing vasodilators, optionally in combination with conventional anti-pyretics.

### BACKGROUND OF THE INVENTION

The white cells of the body produce a substance called interleukin-1 when they digest a germ. Interleukin-1 induces the formation of prostaglandins. Prostaglandins E2 (PGE2) are substances that act on the hypothalamus resetting the body thermostat to a higher level—resulting in a fever. PGE2 is the ultimate mediator of the febrile response. The set-point temperature of the body will remain elevated until PGE2 is no longer present.

Fever is one of the body methods of fighting pathogens. Hence, there is no substantial medical reason to treat fever under 38 degrees Celsius (° C.), though personal sense of comfort may improved by reducing any fever.

The present state of the art of anti-pyretic treatment is based on oral application of medicine. There are four basic categories of medications: aspirin, ibuprofen, acetaminophen, and naproxen. These drugs have broad systemic activity and act as analgesic, anti-inflammatory, and anti-pyretic drugs (i.e., used to relieve pain, inflammation, swelling, and reduce fever).

Aspirin and other non-steroidal anti-inflammatory drugs (NSAID) target a group of enzymes called Cyclooxygenases. These enzymes catalyze a key step in the synthesis of prostaglandins. Prostaglandins are hormones that carry local messages to neighbouring cells (most other hormones carry messages throughout the body). There are two cyclooxygenase genes in humans; the enzymes they make are called COX-1 and COX-2. Put simply, present anti-pyretic medications work as enzyme inhibitors. They interfere with the activity COX-1 and COX-2 enzymes.

COX-1 makes prostaglandins that are necessary for the synthesis of protective gastric mucus in the stomach and for proper blood flow in the kidneys. It also makes a prostaglandin necessary for platelet cell functioning. So by inactivating this enzyme such medications have a negative effect on the stomach and kidneys but a beneficial effect on the circulatory system.

COX-2 makes prostaglandins that are involved in inflammation, pain, and fever. By inhibiting this enzyme, medication can reduce each of these three responses within our bodies.

From the above description it would seem that a better pain-killer than aspirin would be one that inhibited COX-2 but did not inhibit COX-1. Indeed, drugs with these properties have been developed and are referred to as selective COX-2 inhibitors. COX-2 inhibitors such as Celebrex (celecoxib, made by Pfizer) and Vioxx (rofecoxib, made by Merck & Co.) were introduced in 1999. They decrease pain, fever, and inflammation with no negative effects on the stomach. Its world-wide sales were $2.5 billion (US) in 2003. Unfortunately, patients who were on Vioxx for more than. 18 months began to show an increased frequency of serious cardiovascular problems. Vioxx was withdrawn from the market. It is not clear why Vioxx causes cardiovascular problems.

Thus, there is a need for safer methods of fever reduction. In particular, there is advantage for methods which reduce fever without adverse effects on internal organs in general and without significantly affecting the levels of COX-1 enzymes in particular.

Moreover, there is an advantage to novel anti-pyretic use of substances that have already proven their relative safety in various dosages of administration for other indications.

The anti-pyretic effect of the present medication is gradual and reaches maximum effect about 2 hours from time of administration.

Therefore, there is absence of, and a need for a treatment that primarily acts to reduce fever without other broad systemic consequences. This is particularly relevant for use in children, where the anti-pyretic activity is the prominent objective of medicinal use of the medicine of the present invention by the consumer public.

Moreover, due to side effects of the drugs currently available on the market, the medical recommendation is of a minimum of 4 hours before repeated use of acetaminophen, and 6 hours between administration of ibuprofen, and to avoid use for more than 3 consecutive days. Hence, it would be advantageous to have a fever treatment which can be safely used for longer periods and shorter intervals.

For young hildren the oral administration of medicine is frequently inconvenient due to lack of cooperation from the patients. Therefore, there is a need and an advantage for topical anti-pyretic treatments applied to the skin.

When the present anti-pyretic medication is applied to patients with high fever, e.g., above 39° C., fever is commonly not restored fully back to normal but instead levels off at lower fever (e.g., at 38° C.). This situation inclines many parents to infer that the fever medication was not fully effective, and may induce them to administer additional doses of medications counter to the prescribed safety instructions. Therefore, there is need and advantage to fever medication with increased effectiveness to reduce fever more down to normal from high levels.

Over 40% of pediatric OTC acetaminophen sales are within Cold&Cough combination formulas; which contain multiple substances including a decongestant, cough suppressant, antihistamine, with the anti-pyretic/analgesic acetaminophen component.

A survey of pediatricians, conducted at the recent Annual Meeting of the American Academy of Pediatrics [American Academy of Pediatrics Convention Study, Oct. 31, 2000, Prepared by Wirthlin Worldwide], shows that 61% of respondents are very concerned that, by combining common over-the-counter cold and fever medications, parents may unwittingly give their children an overdose of medication, putting children's health at risk. The pediatricians' chief concern was that children may be getting an overdose of fever reducers, which can affect the liver or the kidney. Often, parents who are unaware of these ingredients may give their children an additional fever-reducing medication. Therefore, when recommending an over-the-counter cold remedy for their patients, 73% of pediatricians surveyed considered it very important to eliminate the anti-pyretic component from recommended present pediatric cold medication.

Therefore, there is a need and advantage to introduce combination cold&cough medicinal formulas that do not contain NSAID elements (such as acetaminophen or ibuprofen), but still do contain an anti-pyretic component element with lesser overdose risks.

Known anti-pyretic NSAID include, but are not limited to ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene, hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Methods of lowering body temperature, even all the way to hypothermia, have been known in the literature by getting large surfaces of the skin in contact with a cold fluid or solid heat sink (e.g., cold water, pads attached to cooling engines, and others).

For reasons that will become clear later on, vasodilators are relevant to the present invention. Hence, we elaborate below on the present art and use of vasodilator substances.

### Mechanism of Fever

Heat is produced in the anterior of the body due to its internal working processes. Heat is dissipated out of the body through the skin surface. The heat is conducted from the body inside to the skin surface by the blood circulation. Thus, body temperature is regulated by the flow of blood to the skin.

The mechanisms responsible for elevating body temperature in fever condition include: reduction in heat loss by constriction of peripheral vessels whose tone is under control of the sympathetic nervous system; inhibition of panting and sweating, the latter by way of the cholinergic nerves; and increased heat production by means of shivering in voluntary muscles innervated by somatic motor nerves.

### Vitamin B3 Vasodilator Action

Vitamin B3 comes in three principal forms: niacin (nicotinic acid), niacinamide (nicotinamide) and inositol hexaniacinate (inositol hexanicotinate). Each one has its own particular effects when taken in high doses.

Doses of niacin over 50 mg may cause flushing of the skin, lasting about 60 minutes in duration, along with a mild itching sensation and a reddening of the skin. When nicotinic acid is given repeatedly, tolerance to nicotinic acid-induced flushing develops within about a week.

Nicotinic acid can cause vasodilation of cutaneous blood vessels resulting in increased blood flow, principally in the face, neck and chest. This produces the niacin- or nicotinic acid-flush. The niacin-flush is thought to be mediated via the prostaglandin (PG) prostacyclin and via histamine release.

There, appears to be a difference between the control of the wide and small blood vessels, and the associated influence of nitric-oxides. (NO) based vasodilators and B3 vitamin vasodilators. A study in 1994 (John Warren, Vol. 8, February 1994, The FASEB Journal), shows that NO can increase human skin microvascular blood flow in vivo. However, the NO donor sodium nitroprusside was 10,000-fold less potent than PG when injected intradermally. This suggests that on a molar basis endogenous NO has less influence on microvascular flow than PG, the predominant prostaglandin of the microcirculation.

B3 vitamins have been described in multiple therapeutic uses. Niacin (but not niacinamide) can significantly improve cholesterol profile, reducing levels of total and LDL (“bad”) cholesterol and raising HDL (“good”) cholesterol. Therapeutic dosages for such indication are high—around 3000 mg per day continuously over a period of 4 weeks or more. However, unpleasant flushing reactions as well as a risk of liver inflammation and dangerous interactions with other cholesterol lowering drugs have kept niacin from being widely used for this indication.

Niacinamide may improve blood sugar control in both children and adults who already have diabetes. In addition, some evidence had suggested that regular use of niacinamide (but not niacin) might help prevent diabetes in children at special risk of developing it.

Somewhat surprisingly, topical niacinamide has shown some promise for skin conditions like acne, and generally improve skin appearance and elasticity. Niacinamide cream has also shown promise for rosacea.

The inositol hexaniacinate form of niacin (taken orally) may be helpful for intermittent claudication and Raynaud's phenomenon. In addition, weak and in some cases contradictory evidence suggests one of the several forms of niacin might be helpful for people with bursitis, cataracts, HIV infection, schizophrenia, and tardive dyskinesia.

Essentially all of the above uses are associated with regular long term use and administration of therapeutic high doses of B3 vitamins (up to 7000 mg per day). It is indicative of the fact that, in principle, B3 vitamins can be administered over significant periods without prohibitive health consequences. Similarly, the adverse effects known in the literature are a consequence of remarkably prolonged and repeated administration of high dose's B3 vitamins.

It has been recognized that the main vasodilatating effect of B3 vitamins is in the skin extremities of the body. Specifically, a research paper by Morrow et al. in the Journal of Investigative Dermatology Vol. 98 N5 (1992) identifies “the skin as a major site of prostaglandin D₂ release following oral administration of niacin to humans”. I.e., unlike NO-donors whose effect may depend on the intake location, the skin is the major target organ for vasodilatating action of B3 vitamins.

Niacin is used as treatment to reduce cholesterol in patients with such need. One of the known, and undesired, side effects in such patients is an increase in skin temperature and flushing. Curiously, an intake of aspirin 30 minutes before niacin is recommended in order to reduce the flushing side effect. i.e., here aspirin is used to counter an effect of niacin. Niacin effect on the skin due to increase of prostaglandin activity, while Aspirin is a known inhibitor of prostaglandin synthesis. Since ibuprofen has a similar prostaglandin inhibiting effect, it is expected to have the same interaction with B3 vitamins.

Vasodilation induced by topical application of methyl nicotinate was evaluated and compared with the vasodilatory response to acetylcholine and sodium nitroprusside in healthy subjects and diabetic neuropathic patients [Caselli et al. (2003) Topical methyl nicotinate-induced skin vasodilation in diabetic neuropathy, Journal of Diabetes and Its Complications, 17, pp. 205-210]. Ten diabetic patients with peripheral neuropathy and 10 age- and sex-matched healthy control subjects were enrolled. The vasodilatory response to topical application of 1% methyl nicotinate and a placebo emulsion at the forearm and dorsum of the foot skin at 4, 15, 30, 60 and 120 minutes was measured using Laser Doppler Perfusion Imaging. The vasodilatory response to iontophoresis of 1% acetylcholine and 1% sodium nitroprusside solutions was also evaluated. The maximal vasodilatory response to acetylcholine, sodium nitroprusside and methyl nicotinate was similar at the forearm and foot level in the diabetic patients. In the control group, the responses to acetylcholine, sodium nitroprusside and methyl nicotinate were similar on the forearm but in the foot, the methyl nicotinate vasodilatory response was higher when compared to the acetylcholine and sodium nitroprusside responses. Methyl nicotinate-related vasodilation was present 5 minutes after the application, reached its peak at 15-30 minutes and declined to pre-application levels 120 minutes afterwards.

A US Army research group published a paper in 1995 entitled “Increased skin blood flow and enhanced sensible heat loss in human after nicotinic acid ingestion” in the Journal of Thermal Biology volume 20:409-423, and a 1999 US Army report document detailing the same study entitled “Skin Blood Flow Response and Forearm Reactive Hypereamia after Niacin Ingestion”. In this work, skin blood flow following niacin ingestion was examined in healthy adults. Administration by ingestion of a dose of 5 mg niacin per Kg body weight resulted in decrease of core body temperature by 0.44° C. on average, lasting for about 70 minutes. A sharp decrease in body temperature was noticed to begin about 20 minutes after oral ingestion, and the peak of continuous temperature reduction occurred about 50 minutes after ingestion. A significant portion of the subjects reported sever hypotension as an undesired side effect. This research does not teach neither suggests the use of vasodilators such as nicotinic acid or nitroglycerin, at low dosages, for alleviating fever in patients.

### Nitric Oxide (NO) as Vasodilation Agents

Nitric Oxides are natural vasodilators. A variety of Nitric Oxide donors or precursor compounds (NO-donors) are known.

Vasodilation is the widening of blood vessels resulting from relaxation of the muscular wall of the vessels. Vasodilation can alleviate disease and disorders of the cardiovascular system, for example hypertension.

The regulation of blood pressure is a complex event where one of the known mechanisms involves nitric oxide (NO) produced by a dependent form of nitric oxide synthase (NOS). NO produces muscle relaxation in the vessel (dilation).

When the normal level of NO is not produced, either because production is blocked by an inhibitor, or in pathological states, the vascular muscles do not relax to the appropriate degree.

All blood vessels that are surrounded by smooth muscles can dilate in response to changes in NO. However, in general, the large blood vessels respond strongly to NO. As one moves into arterioles, the vessels are more closely linked with tissue beds, these vessels are influenced to dilate not only in response to increased NO production by endothelial cells, but is also in response to regional changes in the levels of other vasodilators, compounds such as adenosine and prostaglandin I2.

An elaboration of NO-donor compounds and their application is given, for example, in U.S. Pat. No. 6,287,601 and U.S. Pat. No. 7,048,951, and the article “Nitric oxide donors and the skin” published in the Journal Clinical Science (2003) 105, 533-535, and references therein. In addition, various natural ingredients decompose into nitric oxides in the body as described in U.S. Pat. No. 6,340,480, and can also be considered as NO-donors.

The vasodilatating effect of NO-donor compounds and resulting increase of blood flow found great use in the enhancement of male sexual stamina.

The most widely used NO-donor is nitroglycerin. Nitroglycerin in medicine, where it is generally called glyceryl trinitrate, is used as a heart medication (in 2% concentration). It is used as a medicine for angina pectoris (ischaemic heart disease) in tablets, ointment, solution for intravenous use, transdermal patches, or sprays administered sublingually.

The principal action of nitroglycerin is vasodilation—that is, widening of the blood vessels. These effects arise because nitroglycerin is converted into nitric oxide in the body. The main effects of nitroglycerin in episodes of angina pectoris are: subsiding of chest pain, decrease of blood pressure, increase of heart rate, fainting or loss of consciousness (side effect that may occur upon change of posture).

A secondary unspecified supporting role of nitroglycerin in potential anti-pyretic activity is noted in US 2006/0100263 where nitroglycerin is noted as a secondary anti-pyretic agent administered in combination formulation with the primary anti-pyretic agent bicifadine.

Recently, nitroglycerine has also become popular in an off-label use at reduced (0.2%) concentration in ointment form as an effective treatment for anal fissure.

An eventual stimulated synthesis of NO was recently discovered as one of the mechanisms of action of Aspirin. This stimulation is quite indirect. Aspirin induces the formation of NO by triggering the synthesis of 15-epi-lipoxin A₄. The mechanism appears to be acetylation of cyclo-oxygenase. NO is then produced by vascular epithelial cells, and not as a chemical transformation of the Aspirin substance itself. Hence, Aspirin cannot be considered chemically as a NO-donor substance.

The indirect NO activation aspect is special to Aspirin, and is not a feature of the action of other NSAID drugs (hence some of the unique medicinal aspects of Aspirin compared with other NSAIDs).

One company has also produced nitro-aspirin, which combines aspirin with a nitric oxide-releasing moiety. The nitric oxide liberated in the stomach protects the stomach mucosa from damage by gastric hydrochloric acid. In contrast, the present invention proposes the indication for the NO donor use to be the same as for the NSAID use.

It has been recognized that skin vasodilation promotes heat transfer. In particular, US 2005/0065583 describes active body cooling using a heat transfer to an absorbing heat exchange device, and the dissipation of the heat from the skin is assisted by use of vasodilators. Yet, it is not recognized that use of vasodilators such as nitroglycerin and niacin in appropriate amounts can on their own suffide for the reduction of fever without resorting to any additional devices or medicaments.

A further aspect of the present invention refers to the use of NO-donor compounds as anti-pyretics. A US Army research paper from 1991, published on http://stinet.dtic.mil/cgibin/GetTRDoc?AD=ADA387041&Location=U2&doc=GetTRDoc.pdf describes the examination of the effect of topically applied nitroglycerin on skin blood flow. However, this was an experiment on 4 healthy adults in which 7.5 mg of nitroglycerin in 2% paste was applied on a forearm skin area of 9 cm*6 cm for 20 minutes treatment followed by cleaning and 15 minute recovery period in which data was taken. Thus, effects were recorded only for 35 min after initial skin application. This research does not teach neither suggests the use of vasodilators such as nicotinic acid or nitroglycerin, at low dosages, for alleviating fever in patients.

### Other Vasodilators

As noted above, peripheral vessels are more closely linked with tissue beds, and thus these vessels are influenced to dilate not only in response to increased NO production by endothelial cells, but also in response to regional changes in the levels of other vasodilators, compounds such as adenosine and prostaglandin I2. The complex mechanisms of these other vasodilator paths are not fully understood.

Such additional vasodilator agents are e.g. Pentoxifylline, Cilostazol, Tolazoline, Phentolamine, Nicergoline, Phenoxybenzamine, and Ergoloid mesylate,

For example, Cilostazol affects both vascular beds and cardiovascular function. It produces non-homogeneous dilation of vascular beds, with greater dilation in femoral beds than in vertebral, carotid or superior mesenteric arteries. Renal arteries were not responsive to the effects of Cilostazol. The mechanisms of the effects of Cilostazol on the symptoms of intermittent claudication are not fully understood. Several of its metabolites are cyclic AMP (cAMP) phosphodiesterase III inhibitors (PDE III inhibitors), inhibiting phosphodiesterase activity and suppressing cAMP degradation with a resultant increase in cAMP in platelets and blood vessels, leading to inhibition of platelet aggregation and vasodilation.

US 2007/037872 describes compositions, products and methods of inducing NO-independent vasodilation. In particular, an effective amount of a flavanol, a procyanidin or a derivative thereof, or an epimer thereof.

### Sweat Inducing (Diaphoretic) Plant Extracts

Definition: diaphoretic plant. An exemplary list of diaphoretic plants can be found in the web page—www.liberherbarum.com/Sn0049.HTM. In the present invention, “diaphoretic plant” indicates any plant selected from the group of plants delineated in this list.

There is an existing tradition to use sweat inducing (diaphoretic) plants for treatment of fever. The use of such plants is usually via oral ingestion in the form of an infusion or other plant extracts.

### Summary of Main Observations on Background Art

Present anti-pyretic medication—aspirin, acetaminophen, and ibuprofen—all involve the inhibition of both COX-1 and COX-2 enzymes. Since COX-1 inhibition is harmful to various digestive system organs, there is a need for anti-pyretic treatments which involve less or no effect on COX-1 enzyme concentration.

While adult use of aspirin, acetaminophen or ibuprofen medication is more commonly for pain relief, the use of such medicine in children is primarily for fever reduction. Children fever medication needs to be administered in measures fitted according to body weight. The ratio of toxic/therapeutic dosages of present fever medications is 2:1, which leaves narrow margins of error to the commonly unsupervised home user. Thus, there is an advantage for use in children of medicine that can achieve the same level of anti-pyretic activity but at reduced dosage when compared to the medications currently in the market, and thereby increase the safety level for common users.

It has been recognized that skin vasodilators promote local heat transfer. In particular, US 2005/0065583 describes active body cooling using a heat transfer to an absorbing heat exchange device, and the dissipation of the heat from the skin is assisted by use of vasodilators. Yet, it is not recognized that use of vasodilators such as nitroglycerin and niacin in appropriate amounts can on their own suffice for the reduction of fever without resorting to any additional devices or medicament substances.

Skin vasodilators such as nitroglycerin are commonly used to assist penetration of topical medicine, potentially including anti-pyretic drugs. Yet it has not been recognized that vasodilators such as nitroglycerin and niacin in appropriate amounts can on their own suffice for the reduction of fever without resorting to any additional medicaments. It appears that all prior art approaches to pharmaceutical treatment of fever focused entirely on the brain as a “thermostat” organ and neglected to consider the functional role of other body organs in the fever phenomenon and its mechanism.

There is no recognition in the literature of Niacin or other B3 vitamins as potential primary pharmaceutical agents for treatment of fever.

Present anti-pyretic medications have, in fact, little or no effect on the temperature of healthy humans. Hence there is no trivial known induction, from healthy humans to fever patients, of anti-pyretic action of any substance.

Accordingly, it is a principal object of the present invention to overcome the disadvantages in the prior art on fever reduction.

All previous art medications affect simultaneously both fever and pain conditions and have side effects on the functioning of the digestion system. This is indicative of the rather unfocused targeting of the active ingredients. In contrast, the present invention aims at providing medication that acts directly to reduce any fever condition, with negligible effects on both the sensation of pain and on the digestive system.

### SUMMARY OF THE INVENTION

The present invention provides vasodilators as effective independent anti-pyretic substances, causing significant temperature reduction (more than 0.5 degrees) which can be achieved by use of safe doses of B3 vitamins or nitroglycerin (and other NO-donors), in the treatment of patients with fever, either without or with other anti-pyretic substances.

There is a long metabolic and chemical chain reaction from the detection of pathogens in the body, sending signals to the brain, and the brain producing its signals and chemical agents to raise body temperature. But the end of it all is the vasoconstriction of blood vessels in the skin (skin ischemia), which reduce heat loss to the environment and lead to increase retention of body heat that cause the rise of body temperature.

Previous art medications cause disturbance of whole body metabolisms by modifying concentration and activation of basic enzymes in the blood circulation. The anti-pyretic effect of these medications is produced by tuning the levels of hormones affecting the “thermostat” brain organ.

In contrast, the novel approach of the present invention is to introduce fever treatment methods of _{A}cting directly or preferentially on the peripheral skin organ to produce vasodilatation there. Thereby, it is anticipated that side effects on other body organs can be reduced in comparison with present art of anti-pyretic medication.

The premise of the present invention is that core body temperature of a human at rest is near 37° C. if the peripheral skin blood vessels are at normal dilatation. Since fever is a result of skin vasoconstriction, the anti-pyretic goal of vasodilator action is to dilate the vessels back to normal, but there is no need to over-dilate them to the state of visible “niacin flush” condition. Thus, with a preferred anti-pyretic dosage of vasodilator according to the present invention, known undesired side effects such as hypotension and marked skin reddening “niacin flush” are avoided.

The present invention introduces a therapeutic purpose use of vasodilator substances to restore normal body temperature down from elevated fever.

Vasodilators act by relaxing the smooth muscles in the walls of blood vessels in the body. Each vasodilator acts through a specific biochemical mechanism and elicits a vasodilatory effect at different concentrations with different kinetics or the dilation and the prolonged periods of dilation following initial exposure.

Examples of vasodilator substances are provided in preferred embodiments of the present invention.

The most preferred embodiment is the use of B3 vitamins, which act primarily to dilate peripheral blood vessels, as anti-pyretic medication according to the present invention.

An advantage of the present invention is that substantial fever reduction can be obtained within 30 minutes of treatment initialization. Yet, the action of the vasodilator substances (B3 vitamins or NO donors) also dissipates fast, within about 90 minutes. Therefore, slow release techniques may need to be applied in order to guarantee fever reduction over extended periods. Extended or delayed release liquid form administration may be achieved through the use of micro-capsule colloid or emulsion or other liquid embedding or hosting of controlled release methods.

Another novel aspect of the present invention is that it introduces a new combination Cold&Cough formula which includes an anti-pyretic effect without containing an NSAID active ingredient (such as acetaminophen or ibuprofen), and hence pertains to introduce a medicinal formula with lesser overdose risks.

Optionally, in some preferred embodiments, the composition of the invention includes the addition of extract of at least one diaphoretic (sweat inducing) plant. Thereby, leading to increased rate of heat loss from the body and consequently enhancing the reduction of fever.

Furthermore, the present invention also provides the combined simultaneous administration of B3 vitamins and NO-donors, which act synergistically to lower body temperature. Therefore, when used together, the respective dosage of each component ingredient can be reduced in comparison with the dosage that is required to when used singularly to achieve a given level of fever reduction.

Synergetic combination formulas: some preferred embodiments of the present invention comprise the use of vasodilator substances, particularly B3 vitamin, in combination with conventional NSAID anti-pyretic drugs such as acetaminophen, aspirin, or ibuprofen. The administration of such combination can be preferentially done via mixing of the ingredients within a single delivery agent such as a pill, a capsule, a liquid, or a topical patch. Said combined usage has several advantages over the existing art, as detailed below:

(a) a vasodilator component, such as B3 vitamins, typically have fast action and thereby contributes to fast anti-pyretic results while the other drug components contribute to extended effect;

(b) the vasodilator component enhances the anti-pyretic action such that body temperature is further reduced from high towards normal body temperature, more than the action of NSAID alone.

(c) The vasodilator can be used as follow-up prolongation of the anti-pyretic effect of present art substances, e.g., B3 vitamin can extend post acetaminophen anti-pyretic action to 6 hours instead of the independent acetaminophen 4 hours of effectiveness. Note that this element is effectively amounting to reduced dosage of overall medication over a 24 hour period, since the frequency of administration is lowered.

(d) The synergistic anti-pyretic action of the composition can enable the use of reduced dosage of each component in order to reach the same level of anti-pyretic effect.

Previous art combinations, associated with use of B3 vitamins for lowering cholesterol, used aspirin to lower the “niacin flush” side effect of high doses of B3 vitamins. Yet acetaminophen was not indicated for such combinations since it has low anti-inflammatory action. In contrast, for the anti-pyretic indication proposed in the present invention it is the combination of Acetaminophen and B3 vitamin which is a preferred combination with lower required effective dosage of B3 vitamin.

B3 vitamin substances can be administered orally, e.g., in capsules or in syrup or liquid suspension. Since B3 vitamins are water soluble, they can administered by topical applications that are readily absorbed by the skin. In particular, administration apparatus can take the form of a patch.

Similarly, NO-donors can be administered in oral ingestion or via topical application. The methods for both modes of administration are well developed in existing art of NO-donors.

Using the skin as the target organ for treatment to reduce fever, there is preference and advantage to topical administration of B3 vitamin substances in the present invention. As mentioned before, the advantage of topical application of anti-pyretic medicine on the skin, the reduction in the concentration of such medicine in other organs, particularly the stomach and brain, compared with administration by ingestion.

Topical application to the skin is a preferred embodiment for use especially in the children population, which is adverse to oral intake of medications.

Moreover, B3 vitamins have some uncomfortable side effects which may be limited in extent through topical application to the skin instead of oral ingestion. Nicotinate esters are suitable candidates for topical applications (in the form of gel, ointment, or patch). They act as pro-drugs, which cross the skin rapidly and, upon enzymatic hydrolysis, release nicotinic acid. This agent triggers increased cutaneous blood flow, at least partly by forming vasodilating prostaglandins. As a consequence of the dilatation of small arterioles, the skin color changes and the level of oxygen in the skin increases. The time when maximal effect is achieved and the duration of vasodilation depend on the concentration of the drug and its chemical structure (nicotinic acid and different esters: methyl, ethyl, hexyl, benzyl, tetrahydrofurfuryl). The rate of rubefacient action, as well as its effectiveness, depends not only on the rubefacient used but also on the carrier in which the rubefacient is applied.

For practical medicinal use in lowering fever, the main advantages of the present invention over previous art include:
(a) A treatment without COX-1 enzyme inhibition.
(b) Significant anti-pyretic action within 30 minutes of administration.
(c) Stronger anti-pyretic action in reducing fever from elevated towards normal body temperature level.
(d) Safety—Safe natural B3 vitamin with other beneficial contributions.
(e) Extended use—Possible for more prolonged use of several days without adverse side effects.
(f) Possible to alternate without counter-indication with other fever medications. Thus enabling treatment at short time intervals that are not recommended for previous art of anti-pyretic medicines.
(g) Reduced dosage—Synergetic anti-pyretic activity of B3 vitamins and NO-donors can be used at reduced dosage of each single component required to achieve the same effect. Thus minimizing side effects, and enabling effective treatment at dosage levels of over-the-counter products.
(h) Reduced dosage—Synergetic anti-pyretic activity of B3 vitamins together with conventional Aspirin or Acetaminophen or Ibuprofen can be used in reduced dosage of any single component required to achieve the same effect.
(i) Topical administration—Since many vasodilator substances are readily absorbed by the skin, administration of anti-pyretic treatment via topical skin application can be implemented. Such topical administration is of advantageous comfort and compliance with young children.
(j) Ability to introduce a combination Cold&Cough formula which includes an anti-pyretic effect without containing an NSAID active ingredient.
(k) An optional inclusion of sweat inducing substances (e.g., diaphoretic plant extracts) in any of the noted compositions leads to a synergetic effect of reducing fever by increase of both skin blood flow and perspiration. The synergetic action enables the use of reduced dosage of each individual active ingredient.

Additional features and advantages of the invention will become apparent from the description of preferred embodiments.

### DETAILED DESCRIPTION OF THE INVENTION

The embodiments herein presented enable the realization of effective administration of medication for lowering fever when applied to humans in need of such treatment. The use of B3 vitamin substances and NO-donor ingredients in compositions intended for use in reducing fever is the principle goal of the present invention.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

In one aspect, the present invention provides a method of alleviating fever in a human subject suffering from a fever condition, said method comprising administering a therapeutically effective amount of a vasodilator substance, or a composition comprising the same to said subject.

Thus, for the purposes of the present invention, vasodilator substances may be defined as new anti-pyretic agents, reducing or tending to reduce fever, or as febrifuges.

As mentioned before, one of the main advantages of the present invention is the use of a medicament for lowering fever without interfering with COX-1 enzyme function, and without adversely affecting their concentration levels in the body.

In one preferred embodiment, said vasodilator substance is vitamin B3 or a derivative thereof.

The use of vitamin B3 as a medicament is an advantage in that vitamin B3 is a natural, non-toxic product, which may be obtained from natural sources. These features respond to current popular trends to avoid “artificial” or “chemical” products, and search for alternative medicine.

In the context of this invention, we refer together and interchangeably to B3 vitamins and associated esters, and to materials that dissolve into B3 vitamins, as “B3 vitamin substances” or “B3 vitamins”.

As referred to herein, fever, also known as pyrexia, is a medical symptom or condition which describes an increase in internal body temperature (core body temperature) to levels which are above normal, particularly in reaction to pathogens present in the body. Besides the higher body temperature, fever is usually accompanied by shivering, chills, and in more severe cases by seizures or convulsions. Normal temperature generally means 37° C. (98.6° F.) in humans, and includes normal fluctuations of about 0.5 degrees due to external conditions, exercise, normal variations among individual persons, as well as variations resulting from different measuring techniques. Average adult normal body temperature when taken by mouth with a thermometer is 37.8° C., or 98.6° F. Normal rectal temperature is approximately 0.5° C. (1° F.) higher than the oral temperature, while the temperature under the armpit (axillary) is slightly lower than the oral temperature.

Fever may also be triggered by other conditions, including inflammations caused by arthritis or leukemia, where the body produces defective and useless white blood cells that cause fever but cannot fight infection, and in heat stroke, where the body's heat regulating mechanism no longer functions properly, due to overexposure to the sun. Hormonal problems or some medications can also cause fever.

Fever, or a fever condition, may be also accompanied by at least one of the following symptoms: headache, stiff neck, confusion, shaking chills, burning or pain with urination, shortness of breath and cough, localized pain, redness or swelling.

As referred to herein, the term “alleviating fever” is the equivalent of “fever relieve”, “reducing body temperature”, “lowering body temperature”, “reducing fever”, “reducing or alleviating fever-related symptoms like shivering, chills, seizures and/or convulsions”.

“Alleviating fever”, in the context of the present invention, also relates to reducing core body temperature by at least 0.5° C. within 40 to 60 minutes from time of administration.

The term “derivative” includes but is not limited to salts, ethers, acids, amides, esters polymorphs, isomers, or complexes thereof and the likes. In addition, this invention further includes hydrates of the compounds defined herein. The term “hydrate” includes but is not limited to hemihydrate, monohydrate, dihydrate, trihydrate and the like.

The most preferred target population for the treatment of fever by the method provided in the present invention is children, which includes a child, a toddler, an infant or a newborn.

In another preferred embodiment of the present invention, said vasodilator substance is a nitric oxide donor, preferably nitroglycerin. The preferred target population for the treatment with NO donors is adults.

Alternatively, as a combination treatment, the present invention provides a method of alleviating fever, in a subject suffering from a fever condition, said method comprising administering a therapeutically effective amount of a vasodilator substance or a composition comprising the same to said subject, in combination with a therapeutically effective amount of an anti-pyretic substance, or a composition comprising the same, wherein said anti-pyretic substance or composition comprising the same is administered before, after or together with said vasodilator substance (the “combination treatment”).

When administered together, the vasodilator and the anti-pyretic may be active agents comprised in the same composition, provided that there are no undesired drug-drug interactions, or may be administered separately but concomitantly.

In one preferred embodiment, said vasodilator substance is vitamin B3 or a derivative thereof.

In another preferred embodiment, said anti-pyretic substance is selected from the group consisting of acetaminophen, acetylsalicylic acid, a non-steroidal anti-inflammatory agent such as ibuprofen, and derivatives thereof.

Administration of said vasodilator substance is preferably via one of oral, topical, sub-lingual, inhalatory, rectal or transdermal routes.

Oral administration may be in the form of one of a pill, a capsule, a trochee, a lozenge, a caplet, a syrup, an emulsion, a suspension liquid, a spray, or a powder. For oral administration, a powder may be dissolved in any pharmaceutically acceptable solvent, e.g. water.

Topical administration may be in the form of one of an ointment, a cream, a gel, a lotion, a powder, a spray, or a transdermal patch.

Rectal administration is in the form of a suppository.

Inhalatory administration may be in the form of a spray, a gas or a vapor.

The introduction of medicine to the body via the skin surface is commonly referred to as topical or transdermal application. The application can be in the form of a gel, ointment, or cream, and each can be applied bare or incorporated within a patch structure, and each may include penetration enhancing agents. Together all these forms of transdermal applications and combinations of them will be interchangeably referred to as “topical forms of application” or “topical applications” or “transdermal application”.

Transdermal delivery is beneficial because the agents are delivered directly into the blood stream, avoiding first-pass metabolism in the liver.

Transdermal delivery can also provide a sustained and consistent delivery of medication, avoiding peaks and valleys in blood levels which are often associated with oral dosage forms and which are usually undesirable. Thus, using transdermal delivery, one can administer lower doses of drug to achieve the same therapeutic effect compared to oral administration.

Examples of present art of transdermal application are described in U.S. Pat. No. 5,762,952, U.S. Pat. No. 719,475, and US 2006/0013866.

In the context of transdermal drug delivery, vasodilatating chemical's (vasodilators) at low doses are commonly used merely to facilitate the transdermal penetration of intended medicinal active ingredients (see US 2006/0013866). In contrast, a novel element of the present invention is the use of high doses of vasodilators as the primary active ingredient in lowering fever.

An elaboration on topical application of various peripheral vasodilators, which can be used also in preferred embodiments of the present invention, is provided in US 2005/0282870. A focused discussion of topical formulations for the transdermal delivery of B3 vitamins is provided in U.S. Pat. No. 6,677,361. There are known esters of B3 vitamins, such as methyl-nicotinate. The use of such esters is particularly advantageous for transdermal delivery of B3 vitamins in gels, creams, or patches.

Administration of the anti-pyretic substance or composition comprising thereof, when not formulated in the same composition together with the vasodilator substance, is preferably oral, employing one of a pill, a capsule, a trochee, a lozenge, a caplet, a syrup, an emulsion, a suspension liquid, a spray, or a powder (which may be mixed with a liquid).

In yet another embodiment of the methods of the invention, the composition is formulated for slow release. The term “slow-release” here applies to any release from of a formulation that is other than an immediate release wherein the release of the active ingredient is slow in nature. This includes various terms used interchangeably in the pharmaceutical context like extended release, delayed release, sustained release, controlled release, timed release, specific release, targeted release, etc.

The term “slow release formulation” is intended to mean a formulation whereby the tablets thereof are coated or uncoated containing excipients or prepared by special procedures which, separately or together, are designed to modify the rate or the place at which the active ingredient is released, as is defined by the US Pharmacopoeia for modified-release tablets.

Sustained release formulation can be achieved by different techniques, such as matrix tablets, erosion tablets, lattice tablets, or by coating of the tablet or the active ingredient.

Sustained release formulations for oral use may be constructed to release the vasodilator, or the vasodilator in combination with the anti-pyretic, by controlling the dissolution of the vasodilator and/or the anti-pyretic, its diffusion or both. Dissolution or diffusion controlled release may be achieved by appropriate coating of a tablet capsule, pellet or granulate formulation of the vasodilator, or the vasodilator in combination with the anti-pyretic.

The matrix principle is achieved by mixing the active ingredient with hydrocolloid macromolecular excipients in large amounts, typically more than 25%. When ingested, the tablet forms a highly viscous gelatinous mass at the surface maintaining the shape of the tablet. The active component is slowly released from the surface of the gelatinous mass, at a rate which is controlled by its diffusion through the gel-barrier.

The following macromolecular excipients can be used for creating this gel: methylcellulose, hydroxypropyl methylcellose, carboxymethyl starch or other modified cellulosic substances, hydrophilic gums such as pectinates or alginates.

Erosion tablets differ from the matrix tablet in that the excipients used are lipids, which will not dissolve or gel in the stomach, but slowly be eroded, thus releasing the active ingredient. The following lipids are frequently used for this purpose: stearic acid, glycerol monostearate, stearyl alcohol, cetyl alcohol, and hydrogenated fats.

Lattice tablets differ from the former types in that the excipient chosen is insoluble in the stomach. The tablet will therefore not disintegrate, and the active ingredient is released by diffusion, leaving the lattice unchanged. As excipients for lattice tablets, polyvinyl acetate, polyvinyl chloride or polyethylene may be used.

As stated, the sustained release effect can also be achieved either by coating the tablet or by coating the active particles or pellets made herefrom (micros encapsulation). The coating must be made of an insoluble polymer, whereby the active ingredient must traverse by diffusion. As polymers for film coating, ethyl cellulose, polymethacrylates or lipids may be used. Alternatively, a sustained release coating may be selected from coatings comprising cellulose derivatives such as hydroxypropyl methylcellulose, methylcellulose, methylhydroxycellulose, methylhydroxyethyl cellulose, hydroxypropyl cellulose, carboxymethylcellulose, cellulose acetate, cellulose propionate, cellulose butyrate, cellulose valerate, cellulose acetate propionate and cellulose acetate butyrate; acrylate polymers such as acrylic resins, polymethylacrylate, methylmethacrylate, 2-hydroxymethacrylate, polyethylene glycol methacrylate, methacrylate hydrogels; vinyl polymers such as polyvinyl chloride, polyvinyl acetate, vinyl pyrrolidine, polyvinyl pyrrolidone, polyvinyl formal, polyvinyl butyryl, vinyl chloride-vinyl acetate co plymer, vinyl chloride-propylene-vinyl acetate copolymer; silicon polymers such as ladder polymer of sesquiphenyl siloxane and colloidal silica; waxes such as shellac, beeswax, glycowax, castor wax, beef tallow, whale wax, parrafin wax, and canauba wax; stearic acid derivatives and esters such as stearyl alcohol, glyceryl monostearate, glyceryl distearate, glycerol palmitostearate; myristic acid derivatives and esters, palmitic acid derivatives and esters, behinic acid derivatives and esters, dl-polylactic acid; polyethylene; and/or 1,3-butylene glycol.

The coating may be admixed with various excipients such as plasticizers and anti-adhesives such as colloidal silicum dioxide, flavouring agents, lubricating-agents and pigments in a manner known to the person skilled in the art.

Tablet strengthening agents, such as silica, may also be added to the formulation as may binding agents, inert fillers, flavouring agents or lubricating agents.

The vasodilatation effect of B3 vitamins lasts for about an hour, starting minutes after administration. Hence, for extended effect, single dosage applications would have to be repeated after each one hour period. Alternatively, a single application containing a higher dosage delivered by slow release may be preferred.

The methods of treatment described in the present invention provide that core body temperature is reduced by at least 0.5° C. within 40 minutes from time of administration of the vasodilator or composition comprising the same.

As referred to herein, paracetamol or acetaminophen is the active metabolite of phenacetin, a so-called coal tar analgesic. It is a major ingredient in numerous cold and flu medications, including Tylenol and Panadol, among others. It is considered safe for human use at recommended doses. The words acetaminophen and paracetamol come from the chemical names for the compound: para-acetylaminophenol and para-acetylaminophenol (the brand name Tylenol also derives from this name: para-acetylaminophenol). In some contexts, it is shortened to APAP, for N-acetyl-para-aminophenol.

Aspirin, or acetylsalicylic Acid is an acetyl derivative of salicylic acid that is white, crystalline, weakly acidic substance, with melting point 137° C. As referred to herein, aspirin is also known as 2-(acetyloxy)-Benzoic acid; Solpyron; Ecotrin; Colfarit; Asatylin; Acetophen; Acetosal; Rhodin; o-Acetoxybenzoic Acid; Extren; Benaspir; Entericin; Bialpirinia; Contrheuma Retard; Salicylic Acid Acetate.

The effective dosage of aspirin for adults is of 300 to 1000 mg, generally taken four times a day for fever or arthritis, with a maximum dose of 8000 mg (8 grams) a day.

Ibuprofen is the shortened name for iso-butyl-propanoic-phenolic acid, a non-steroidal anti-inflammatory drug originally marketed as Brufen, and since then under various trademarks. Effective dosages are between 5-10 mg per kg.

The therapeutic doses that need to be administered to achieve a given reduction of fever are generally proportional to body weight. Therefore, in some preferred embodiments of the present invention there will be a clear separation between packaged doses for use by adults and by children.

Alternatively, preferred embodiments of the present invention may consist of a given unit of minimal dose, which in turn can be administered in fixed multiples dependent on the user body weight.

The effective dosage of acetaminophen is between 5 mg to 40 mg per body weight of said subject to be treated. Thus, said dosage of acetaminophen may be 5, 7.5, 10, 12.5, 15, 17.5, 20, 22.5, 25, 27.5, 30, 32.5, 35, 37.5 or 40 mg per kg.

In another preferred embodiment of the present invention, for reducing fever of 38° C. degrees or more by at least 0.5 degrees on average, the dosage of vitamin B3 is equivalent to between 0.1 mg to 4 mg niacin per Kg body weight of user, per hour. Thus, the dosage of vitamin B3 may be 0.1, 0.2, 0.3, 0.4, 0.5, 0.6, 0.7, 0.8, 0.9, 1.0, 1.1, 1.2, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg per kg per hour.

In one preferred embodiment of the present invention, for reducing fever of 38 degrees or more by at least 0.5 degrees on average, the dosage of NO-donors is equivalent to between 0.02 mg to 0.2 mg nitroglycerins per Kg body weight of user per hour. Thus, the dosage of NO-donor may be 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2 mg per kg per hour.

In terms of preferred dosage of vitamin B3, the present invention more preferably stipulates the use of less than 2 mg per 1 Kg body weight. For anti-pyretic purpose, the rule of thumb for the ideal effective dosage of B3 vitamin is that—if a “niacin flush” is significantly visible then the dosage is unnecessarily too high. Fever physiologically develops by constriction of the skin blood vessels. The B3 vitamin therapeutic action should be to return vessels dilation to near normal, and not to over dilate them (i.e., avoid creating the flush and uncomfortable tingling sensation).

In particular, an optimal dosage release is such that it will not cause a substantially noticeable “red flushing” of the skin due to dilatation of the skin blood vessels substantially beyond normal level. It is expected that such choice of dosage also avoids the uncomfortable tingling sensation while remaining effective for fever reduction.

In another preferred embodiment of the present invention the administered treatment is a combination of NO-donors and B3 vitamins in dosages equivalent to between 0.02 mg and 0.1 mg of nitroglycerin and between 0.2 mg to 2 mg of niacin respectively per kg body weight per hour.

In some preferred embodiments, the administration of the B3 vitamin substances and/or NO-donors is done topical by patch delivery methods such as known in the art.

In some preferred embodiments, the administration of the principle B3 vitamin substances and/or NO-donors is done oral within flavored syrup. Preparation methods of such flavored syrups, particularly for use by children, are known in the art.

In some preferred embodiments, the administration of the principle B3 vitamin substances and/or NO-donors is assisted by skin penetration enhancing delivery methods such as known in the art.

In some preferred embodiments, the administration of the principle B3 vitamin substances and/or NO-donors is done by extended release delivery methods such as known in the art.

US 2006/0013866 discusses the use of ibuprofen in combination with a vasodilator where the vasodilator is use in low dose as a penetration enhancer, where said vasodilator being present in an amount of less than 1% w/w. In contrast, the present invention introduces the vasodilator as a medicinal active ingredient at high dose, of typically more than 10% w/w, and is meant to reduce the medicinally required dose of ibuprofen.

According to the methods of the invention, the effective dosage of vitamin B3 is between 0.1 mg to 4 mg per body weight of said subject to be treated per hour.

Thus, said dosage of vitamin B3 may be 0.1, 0.2, 0.3, 0.4, 0.5, 0.6, 0.7, 0.8, 0.9, 1.0, 1.1, 1.2, 1.2, 1.3, 1:4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg per kg per hour.

As referred to herein, the term “effective dosage” or “effective amount” means an amount necessary to achieve a selected result, which at present, involves the amount of vasodilator, or an anti-pyretic vasodilator in combination with an anti-pyretic, or the amount of a composition (or compositions) comprising thereof necessary for treating or alleviating fever, or for lowering core body temperature.

Said therapeutic effective amount, or dosing, is dependent on severity and responsiveness of the disease state to be treated, with the course of treatment lasting one hour to several hours, or until a cure is effected or a diminution of the disease state (i.e. fever) is achieved. Persons of ordinary skill can readily determine optimum dosages, dosing methodologies and repetition rates. Optimum dosages may vary depending on the relative potency of individual vasodilators of the invention, or compositions comprising thereof, and can generally be estimated based on EC₅₀, found to be effective in in vitro as well as in in vivo animal models. Persons of ordinary skill in the art can easily estimate repetition rates for dosing based on measured residence times, concentrations, and adjustment to the employed vasodilator.

The terms “treat, treating or treatment” as used herein mean ameliorating one or more clinical indicia of disease activity in a patient having fever or a fever-inducing condition. “Treatment” refers to therapeutic treatment.

By “patient” or “subject in need” is meant any mammal for which fever treatment is desired in order to overcome said higher than normal core body temperature, particularly a human subject.

The term “children” includes newborns, infants, toddlers and 3 year-olds and older children.

Usually, a “therapeutically effective amount” is also determined by the severity of the disease in conjunction with the preventive or therapeutic objectives, the route of administration and the patient's general condition (age, sex, weight and other considerations known to the attending physician).

Said composition comprising the vasodilator optionally further comprises a diaphoretic plant extract substance, or a composition comprising the same.

In the present invention, “diaphoretic plant” is referred to as indicating any plant selected from the group of plants delineated in an exemplary list of diaphoretic plants which can be found in the web page—www.liberherbarum.com/Sn0049.HTM , and is detailed below:

Abelmoschus esculentus, Abies alba, Abronia fragrans, Acanthospermum australe, Acanthospermum hispidum, Achillea millefolium, Achillea moschata, Achillea ptarmica, Achillea sibirica, Achyrocline satureioides, Acinos alpinus, Aconitum chasmanthum, Aconitum chinense, Aconitum ferox, Aconitum fischeri, Aconitum kusnezoffii, Aconitum lycoctonum, Aconitum napellus, Aconitum orientale, Aconitum uncinatum, Aconitum volubile, Acorus calamus, Acorus gramineus, Adansonia digitata, Adiantum capillus veneris, Adiantum raddianum, Adiantum trapeziforme, Aegiphila sellowiana, Agastache foeniculum, Agastache rugosa, Agave americana, Ageratina aromatica, Agropyron repens, Akebia quinata, Alhagi mannifera, Alhagi maurorum, Alisma plantago-aquatica, Alliaria officinslis, Allium ampeloprasum, Allium ampeloprasum babbingtonii, Allium porrum, Allium sativum, Allium sativum var. orphioscorodon, Allium urisnum, Alnus rhombifolia, Aloysia gratissima, Alternanthera brasiliana, Althea officinalis, Amaranthus spinosus, Amburana cearensis, Amelanchier alnifolia, Anagallis arvensis, Anatherum muricatum, Anchusa azurea, Andradea floribunda, Andropogon bicornis, Andropogon leucostachyus, Andropogon nardus, Anemia tomentosa, Anemone decapetala var. foliosa, Angelica anomala, Angelica archangelica, Angelica archangelica ssp. Litoralis, Angelica atronpurpures, Angelica dahurica, Angelica silvestris, Angelica sinensis, Annona squamosa, Anonymos sempervirens, Anthemis cotula, Anthriscus cerefolium, Apocynum androsaemifolium, Apocynum cannabinum, Aquilegia brevistyla, Aquilegia caerulea, Aquilegia canadensis, Aquilegia flavescens, Aquilegia formosa, Aquilegia formosa ssp. Truncata, Aquilegia jonesii, Aquilegia pubescens, Aquilegia shockleyi, Aquilegia vulgaris, Aralia californica, Aralia hispida, Aralia nudicaulis, Aralia racemosa, Aralia spinosa, Arctium lappa, Arctium minus, Argemone mexicana, Arisaema triphyllum, Aristolochia arcuata, Aristolochia clausenii, Aristolochia clematitis, Aristolochia cymbifera, Aristolochia reticulata, Aristolochia serpentaria, Aristolochia tomentosa, Aristolochia triangularis, Arnica chamissonis, Arnica montana, Artemisia abrotanum, Artemisia tripartita, Artemisia vulgaris, Arum maculatum, Arundo donax, Asarum canadense, Asarum europaeum, Asarum heterotropoides, Asarum sieboldii, Asclepias curassavica, Asclepias syriaca, Asclepias tuberosa, Asparagus adscendens, Asparagus officinalis, Asperula odorata, Asplenium scolopendrium, Atherosperma moschatum, Atropa belladonna, Aurantium var. Citrus, Avicennia schaueirana, Baccharis trimera, Ballota nigra, Balsamorhiza sagittata, Bells perennis, Berberis aristata, Berberis vulgaris, Betula lenta, Betula pendula, Betula pubescens, Blanchetia heterotricha, Boletus laricis, Bomarea edulis, Borago officinalis, Borosma sp., Borreria poaya, Botrychium virginianum, Bouchea fluminensis, Bowdichia virgilioides, Brassica nigra, Broussonetia papyrifera, Bryonia dioica, Buddleja brasiliensis, Buddleja brasiliensis ssp. stachyoides, Bupleurum chinense, Bupleurum fakatum, Buxus sempervirens, Buxus wallichiana, Cacalia sonchifolia, Calamintha grandiflora, Calamintha nepeta, Calamintha sylvatica, Calendula arvensis, Calendula officinalis, Calluna vulgaris, Calotropis procera, Caltha palustris, Camellia sinensis, Camphorosma monspeliaca, Campsis radicans, Canna glauca, Canna indica, Capraria biflora, Capsicum annuum, Capsicum frutescens, Cardiospermum halicacabum, Carex arenaria, Carlina acaulis, Carlina vulgaris, Carthamnus lanatus, Carthamus tinctorius, Caryocar villosum, Casearia obiqua, Casearia sylvestris, Caulophyllum robustum, Caulophyllum thalictroides, Cedrus deodara, Celastus dependens, Celastrus scandens, Centaurea nigra, Centaurea scabiosa, Centaurium erythraea, Cephaelis ipecacuanha, Cephalis ruelliaefolia, Cephalanthus occidentalis, Cestrum corymbosum, Cestrum laevigatum, Cestrum parqui, Chaenorrhinum minus, Chamaemelum nobile, Chamomilla recutita, Chamomilla suaveolens, Chaptalia integerrima, Chelidonium majus, Chenopodium ambrosioides, Chenopodium ambrosioides anthelminticum, Chenopodium multifidum, Chimaphila maculata, Chiococca alba, Chondrodendron filipendulum, Chrysanthemum leucanthemum, Chrysanthemum vulgare, Cimicifuga dahurica, Cimicifuga foetida, Cimicifuga racemosa, Cinnamomum aromaticum, Cinnamomum camphora, Cinnamomum verum, Cirsium nipponicum, Cochrocentrum, Cissampelos fasciculata, Cissampelos glaberrima, Cissus sicyoides, Cissus verticillata, Citrus aurantiifolia, Citrus limon, Citrus sinensis, Clematis recta, Cleome viscosa, Clinopodium vulgare, Cnicus benedictus, Coleus amboinicus, Collinsonia canadensis, Corallorhiza odontorhiza, Cornus alternifolia, Cornus florida, Corylus avellana, Costus spicatus, Costus spiralis, Coumarouna odorata, Crocus nudiflorus, Crocus sativus, Crotalaria stipularia, Cullen corylifolium, Cunila origanoides, Cunila spicata, Cuphea aperta, Cuphea carthagenensis, Cuphea ingrata, Cuphea lutescens, Cuscuta chinensis, Cuscuta japonica, Cymbopogon citratus, Cynanchum vincetoxicum, Cynodon dactylon, Cyperus esculentus, Cyperus rotondus, Cypripedium calceolus parviflorum, Cypripedium calceolus pubescens, Daphnopsis brasiliensis, Debregeasia edulis, Dendranthema x grandiflorum, Desmodium oxyphyllum, Dianthus caryophyllus, Dianthus chinensis, Dicentra cucullaria, Diodia brasiliensis, Dioscorea mexicana, Dioscorea villosa, Diphylleia cymosa, Dipsacus fullonum, Dipsacus pilosus, Dipteryx alata, Dipteryx oppositifolia, Dodonaea viscosa, Dorema ammoniacum, Dorstenia arifolia, Dorstenia asaroides, Dorstenia brasiliensis, Dorstenia cayapia, Dorstenia contrajerva, Drosera rotundifolia, Dryobanalops aromatica, Dryopteris cristata, Echinacea angustifolia, Echinacea pallida, Echium vulgare, Elephantopus micropappus, Elephantopus mollis, Elephantopus scaber, Eleusine indica, Elsholtzia ciliata, Ephedra, Ephedra distachya, Ephedra equisetina, Ephedra gerardiana, Ephedra intermedia, Ephedra major, Ephedra triandra, Equisetum arvense, Equisetum hiemale, Erigeron annuus, Erigeron philadelphicus, Eryngium aquaticum, Eryngium campestre, Eryngium foetidum, Eryngium maritimum, Eryngium yuccifolium, Erythrina velutina, Erythroxylum coca, Eschscholzia californica, Esenbeckia leiocarpa, Eucalyptus globulus, Eugenia uniflora, Eugenia uruguayensis, Eupatorium cannabinum, eupatorium fortunei, Eupatorium maculatum, Eupatorium perfoliatum, Eupatorium triplinerve, Ferula narthex, Filipendula hexapetala, Filipendula ulmaria, Fleurya aestuans, Fraxinus excelsior, Fraxinus nigra, Fuinaria officinalis, Galega officinalis, Galium aparine, Galium boreale, Galium verum, Genista germanica, Genista trinctoria, Geum aleppicum, Geum riva,Le, Geum urbanum, Gillenia stipulata, Gillenia trifoliata, Glechon ciliata, Glechon spathulata, Glycine max, Gnaphalium cheiranthifolium, Gnaphalium luteo-album, Gnaphalium uliginosum, Gnetum urens, Guaiacum officinale, Guazuma ulmifolia, Gypsophila arrostii, Gypsophila struthium, Hedeoma pulegioides, Hedera helix, Hedera nepalensis, Heimia myrtifolia, Heimia salicifolia, Helichrysum stoechas, Heliotropium amplexicaule, Herreria salsaparilha, Hesperis matronalis, Hieracium pilosella, Hydrangea arborescens, Hypitis facilulata, Hypitis homalophylla, Hypitis suaveolens, Hypitis tomentosa, Hypitis umbrosa, Hyptis atrorub ens, Hyptis crenata, Hyptis umbrosa, Hyssopus officinalis, Ilex aquifolium, Ilex paraguariensis, Imperata brasiliensis, Imperatoria ostruthium, Inula helenium, Jacaranda brasiliana, Jacaranda copaia, Jasminum nudiflorum, Juniperus californica, Juniperus communis, Juniperus communis nana, Juniperus communis ssp. alpina, Juniperus horizontalis, Juniperus scopulorum, Juniperus silicicola, Juniperus virginiana, Justicia gendarussa, Justicia procumbens, Kuhnia eupatorioides, Kyllingia odorata, Lamium amplexicaule, Lamium purpureum, Lantana camara, Lantana montevidensis, Lantana undulata, Laplacea fruticosa, Laurus nobilis, Lavandula angustifolia, Ledebouriella seseloides, Ledum colombianum, Ledum glandulosum, Ledum groenlandicum, Ledum palustre, Leonotis nepetifolia, Leonurus cardiaca, Leonurus sibiricus, Leucas martinicensis, Levisticam officinale, Liatris spicata, Ligusticum brachylobum, Ligusticum jeholense, Ligusticum porteri, Ligusticum sinense, Ligusticum lucidum, Lindera benzoin, Lindera stynchnifolia, Lobelia dortmanna, Lobelia inflata, Lobelia siphilitica, Lomatium nudicaule, Lonicera periclymenum, Luxemburgia polyandra, Lychnophora ericoides, Lychnophora rosmarinifolia, Lycopsis arvensis, Machaerium declinatum, Magnolia acuminata, Magnolia denudata, Magnolia glauca, Magnolia grandiflora, Marjorana hortensis, Marrubium vulgare, Melaleuca alternifolia, Melaleuca hypericifolia, Melampodium divaricatum, Melanoxylon brauna, Melissa officinalis, Mentha aquatica, Mentha arvensis, Mentha arvensis piperascens, Mentha cunninghamia, Mentha diemenica, Mentha pulegium, Mentha x piperita citrata, Mentha x piperita officinalis, Mentha x piperita ssp. Vulgaris, Menyanthes trifoliata, Miconia theaezans, Microgramma squanulosa, Microgramma vaccinifolia, Mikania glomerata, Minyranthus heterophylla, Monarda didyma, Monarda menthifolia, Monarda punctata, Moniera trifolia, Morus alba, Morus alba multicaulis, Muhlenbergia asperifolia, Nepata cataria var. citrodora, Nepeta cataria, Nerium oleander, Nicandra physalodes, Nigella sativa, Ocimum basilicum, Ocimum canum, Ocimum fluminense, Ocimum nudicaule, Ocimum selloi, Ocotea teleiandra, Oenanthe aquatica, Origanum onites, Origanum vulgare, Origanum vulgare hirtum, Origanum x majoricum, Ormosia arborea, Ormosia monosperma, Ottonia anisum, Packera aurea, Palicourea rigida, Papaver argemone, Papaver dubium, Papaver nudicaule, Papaver, orientale, Papaver somniferum, Parkinsonia aculeata, Passiflora incarnata, Paulinia cupana var. sorbilis, Paulinia pinata, Paullinia cupana, Perilla frutescens, Perilla frutescens nankinesis, Pessopteris crassifolia, Petasites hybridus, Petiveria alliacea, Petrea insignis, Photomorphe umbellata, Phyllanthus niruri, Physalis angulata, Picea abies, Pilocarpus jaborandi, Pilocarpus microphyllus, Pilocarpus pennatifolius, Pilocarpus spicatus, Pimpinella anisum, Pimpinella major, Pimpinella saxifraga, Pinus roxburghii, Piper arboreum, Piper jaborandi, Piper mar ginatum, Pithecoctenium crucigerum, Plantago psyllium, Poiretia tetraphylla, Polemonium caeruleum, Polemonium reptans, Polygala amarella, Polygala senega, Polygala vulgaris, Polygonum hydropiper, Polypodium lepidopteris, Populus nigra, Populus tremuloides, Porophyllum obscurum, Poterium sanguisorba, Primula acaulis, Primula veris, Prunus spinosa, Pseudognaphalium obtusifolium, Psoralea glandulosa, Pteridium aquilinum, Pteridium fraxinnifolia, Pueraria lobata, Pueraria montana chinensis, Pueraria pseudohirsuta, Pulmonaria maculosa, Pulsatilla pratensis, Pulsatilla vulgaris, Pycnanthemum albescens, Pycnanthemum flexuosum, Pycnanthemum incanum, Pycnanthemum virginianum, Ranunculus acer, Ranunculus bulbosus, Ranunculus sceleratus, Remirea maritina, Reseda odorata, Rhabdocaulon denudatus, Rhododendron anthopogon, Rhododendron ferrugineum, Ribes nigrum, Rosmarinus officinalis, Rubus idaeus, Ruscus aculeatus, Ruta graveolens, Salix, Salix alba, Salix alba caerulea, Salix alba var. vitellina, Salix gooddingii, Salix lasiolepis, Salix nigra, Salix purpurea, Salix purpurea lambertiana, Salvia lyrata, Sambucus australis, Sambucus caerulea, Sambucus canadensis, Sambucus ebulus, Sambucus nigra, Sambucus nigra laciniata, Sambucus racemosa, Sambucus racemosa kamtschatica, Sambucus wightiana, Sambucus williamsii, Sanguisorba officinalis, Saponaria officinalis, Sassafras albidum, Satureja calamintha, Scaevola plumieri, Schizachyrium breviofolium, Scleria hirtella, Scrophularia marilandica, Scrophularia nodosa, Senecio erucifolius, Senecio jacobaea, Senecio vulgaris, Senna alata, Senna occidentalis, Senna uniflora, Silphium perfoliatum, Silybum marianum, Sinapis alba, Siparuna cujabana, Siparuna erythrocarpa, Siparuna guianensis, Siparuna limoniodora, Sison amomum, Sisyrinchium vaginatum, Smilax aspera, Smilax china, Smilax longifolia, Smilax papyracea, Solanum dulcamara, Solanum nigrum, Solanum paniculatum, Solanum scabrum, Solidago odora, Solidago virgaurea, Sonchus asper, Sophora tomentosa, Sphaerocionium aureum, Spigelia flemmingiana, Spigelia glabrata, Spigelia humboldtiana, Spiro dela polyrhiza, Stachys arvensis, Stachytarpheta cayennensis, Stachytarpheta jamaicensis, Streptopus roseus, Spratensis, Swartzia tomentosa, Symplocarpus foetidus, Syzygium aromaticum, Syzygium cumin, Tagetes minuta, Tagetes patula, Taraxacum officinale, Taxus baccata, Taxus brevifolia, Taxus canadensis, Tephrosia virginiana, Tetracera aspera, Tetracera oblongata, Tetracera sellowiana, Tetracera volubilis, Teucrium canadense, Teucrium chamaedrys, Teucrium cubense, Teucrium scordium, Teucrium scorodonia, Thlaspi arvense, Thuja occidentalis, Thymus praecox ssp. arctica, Thymus serpyllum, Thymus vulgaris, Tilia, Tilia americana, Tilia amurensis, Tilia caroliniana, Tilia chinensis, Tilia cordata, Tilia cordata x platyphyllos, Tilia heterophylla, Tilia japonica, Tilia mongolica, Tilia oliveri, Tilia platyphylla,l Tilia tomentosa, Tilia tuan, Torreya californica, Trashyspermum ammi, Tragia volubilis, Trieste perfoliatum, Tsuga canadensis, Tsuga caroliniana, Tsuga chinensis, Tsuga heterophylla, Tsuga mertensiana, Tussilago farfara, Urtica gracilis, Vaccinium myrtilloides, Valeriana hardwickii, Veratrum viride, Verbascum densiflorum, Verbascum nigrum, Verbascum thapsus, Verbena hastata, Verbena officinalis, Veronica beccabunga, Veronica chamaedrys, Veronica officinalis, Viburnum cassinoides, Viola biflora, Viola odorata, Viola tricolor, Vitex agnus castus, Vitex cannabifolia, Waltheria communis, Waltheria douradinha, Wedelia minor, Wyethia angustifolia, Wyethia mollis, Xanthium strumarium, Xanthoxylum americanum, Xanthoxylum bungeanum, Xanthoxylum fagara, Xanthoxylum tingoassuiba, Zanthoxylum simulans.

Preferably said diaphoretic plant extract substance is not one which is a direct precursor for the production of salicylic acid. In one non-limiting example, said plant extract is obtained from the plant Capsicum oleoresin.

An additional vasodilator substance, or a composition comprising the same, may be comprised in the composition for alleviating fever, said substance being selected from the group consisting of arginine, bencyclane fumarate, benzyl nicotinate, buphenine, histamine, hydrochloride, ciclonicate, cyclandelate, ethyl nicotinate, hepronicate, hexyl nicotinate, hydralazine, inositol nicotinate, isoxsuprine hydrochloride, methyl nicotinate, minoxidol, naftidrofuryl oxalate, nicametate citrate, niceritrol, nicoboxil, nicofuranose, nicotinyl alcohol, nicotinyl alcohol tartrate, nicotinic acid, niacinamide, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, vitamin E nicotinate, and xanthinol nicotinate; centrally acting vasomodulatory agents including clonidine, quanaberz, and methyl dopa; Alpha-adrenoceptor blocking agents include indoramin, phenoxybenzamine, phentolamine, and prazosin. Adrenergic neuron blocking agents include bedmidine, debrisoquine, and guanethidine; ACE inhibitors including benazepril, captopril, cilazapril, enalapril, fosinopril, lisinopril, perindopril, quinapril, and ramipril; ganglion-blocking agents include pentolinium and trimetaphan; calcium channel blockers including amlodipine, diltiazem, felodipine, isradipine, nicardipine, nifedipine, nimodipine, and verapamil; Prostaglandins including prostacyclin, thrombuxane A2, leukotrienes, PGA, PGA1, PGA2, PGE1, PGE2, PGD, PGG, and PGH; Angiotensin II analogs including saralasin.

Alternatively, the composition comprises yet another vasodilator substance, or a composition comprising the same, said substance being selected from the group consisting of Pentoxifylline, Cilostazol, Tolazoline, Phentolamine, Nicergoline, Phenoxybenzamine, and Ergoloid mesylate.

In another preferred embodiment, Phenoxybenzamine is used as vasodilator. Phenoxybenzamine is a long-acting, adrenergic, alpha-receptor blocking agent which can produce and maintain “chemical sympathectomy” by oral administration. Its effect can last for 24 hours. Phenoxybenzamine increases blood flow to the skin, mucosa and abdominal viscera, and lowers both supine and erect blood pressures. It has no effect on the parasympathetic system. Phenoxybenzamine works by blocking alpha receptors in certain parts of the body. Alpha receptors are present in the muscle that lines the walls of blood vessels. When the receptors are blocked by Phenoxybenzamine, the muscle relaxes and the blood vessels widen. This widening of the blood vessels results in increase blood flow which we stipulate as leading to reduction of fever.

Niacin effects on the skin are due to an increase of prostaglandin activity, while Aspirin is a known inhibitor of prostaglandin synthesis. Since ibuprofen has similar prostaglandin inhibiting effect, it would be expected that ibuprofen would have the same interaction with B3 vitamins as aspirin does. On the other hand, acetaminophen has much less effect on prostaglandin levels in the skin (which is why it is not an effective anti-inflammatory agent). Therefore, the present invention suggests that the effective anti-pyretic dosage of B3 vitamin is lower in combination with acetaminophen than with NSAIDs of the Aspirin and ibuprofen type.

Thus, in another aspect the present invention provides a pharmaceutical composition comprising as active ingredient a vitamin B3 compound and acetaminophen, for oral, sub-lingual, rectal and inhslatory administration.

In a preferred embodiment of said pharmaceutical composition, the ratio (w/w) of said vitamin B3 compound to acetaminophen is between 1:30 and 1:3. Thus, the ratio of vitamin B3 to acetaminophen may be 1:30, 1:28, 1:25, 1:23, 1:20, 1:18, 1:15, 1:12, 1:10, 1:8, 1:7.5, 1:7, 1:6.5, 1:6, 1:5.75, 1:5.5, 1:5.25, 1:5, 1:4.75, 1:4.5, 1:4.25, 1:4, 1:3.9, 1:3.8, 1:3.75, 1:3.7, 1:3.6, 1:3.5, 1:3.4, 1:3.3, 1:3.25, 1:3.2, 1:3.1 or 1:3.0.

The pharmaceutical composition may be formulated as one of a pill, a capsule, a trochee, a lozenge, a caplet, a syrup, an emulsion, an aqueous or non-aqueous solution or suspension, a powder, a spray and a suppository. When used in the methods of the present invention, the pharmaceutical composition may also be in the form of an ointment, a cream, a gel, a lotion, or a transdermal patch.

Specifically, said pharmaceutical composition is for alleviating fever in a human subject suffering from a fever condition.

The effective dosage of vitamin B3 provided by the pharmaceutical composition is between 0.1 mg to 4 mg per body weight of said subject to be treated per hour. Thus, said dosage of vitamin B3 may be 0.1, 0.2, 0.3, 0.4, 0.5, 0.6, 0.7, 0.8, 0.9, 1.0, 1.1, 1.2, 1.2, 1.3, 1.4, 1.5, 1.6, 1.7, 1.8, 1.9; 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg per kg per hour.

The effective dosage of acetaminophen provided by the pharmaceutical composition is between 5 mg to 40 mg per body weight of said subject to be treated per hour. Thus, said dosage of acetaminophen may be 5, 7.5, 10, 12.5, 15, 17.5, 20, 22.5, 25, 27.5, 30, 32.5, 35, 37.5 or 40 mg per kg.

Said pharmaceutical composition may further comprise a pharmaceutically acceptable adjuvant, carrier, excipient or diluent.

In a particular embodiment, said pharmaceutical composition comprising as active ingredient a vitamin B3 compound and acetaminophen is not for topical administration.

Administration of said pharmaceutical composition provided by the invention, results in the reduction of core body temperature by at least 0.5° C. within 40 minutes.

The term “within 40 minutes” is to be understood as an estimate, and, for the purposes of the present invention, includes a time frame varying from 35 minutes up to 60 minutes, and all the fractions in between, including 45, 50 and 55 minutes.

The pharmaceutical composition may further comprise a diaphoretic plant extract substance or a composition comprising the same.

In another embodiment, the pharmaceutical composition further comprises at least one additional therapeutically effective compound, said compound being selected from the group consisting of an anti-histamine, a cough suppressant, a decongestant, an expectorant, a muscle-relaxant, an analgesic, caffeine, an antibiotic, an anti-inflammatory, or any mixture thereof.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein. Conventional pharmaceutical carriers, aqueous, powder or oily bases, thickeners and the like may be necessary or desirable.

Another aspect of the present invention is to provide combination formulas for the treatment of cold or flu symptoms, particularly fever; where the combination formula has a composition which include an effective amount of B3 and comprising a further medicament useful in a cough and/or cold remedy including at least one active ingredient which is an antihistamine, or a cough suppressant, or a decongestant, or an expectorant, or a muscle-relaxant, or caffeine, or an analgesic or a mixture thereof, wherein the effective dosage of B3 is equivalent to between 1 mg to 10 mg niacin per Kg body weight of user.

In another preferred embodiment of the present invention, for reducing fever in combination formulas for the treatment of cold or flu symptoms; where the combination formula has a composition which include an effective amount of NO-donor and comprising a further medicament useful in a cough and/or cold remedy including at least one active ingredient which is an antihistamine, or a cough suppressant, or a decongestant, or an expectorant, or a muscle-relaxant, or caffeine, or an analgesic or a mixture thereof.

Preferred embodiments of the above noted combination cough and/or cold remedie formulas will not contain acetaminophen and neither ibuprofen, yet will be effective in reducing fever by a noticeable amount.

Examples of further medicaments useful in a cough and/or cold remedy, include any ingredient commonly used in a cough or cold remedy, for example, an anti-histamine, caffeine or another xanthine derivative, a cough suppressant, a decongestant, an expectorant, a muscle relaxant, a vitamin and a co-analgesic such as codeine or another NSAID or combinations thereof. Suitable anti-histamines which are preferably non-sedating include acrivastine, astemizole, azatadine, azelastine, bromodiphenhydramine, brompheniramine, carbinoxamine, cetirizine, chlorpheniramine, cyproheptadine, dexbrompheniramine dexchlorpheniramine, diphenhydramine, ebastine, ketotifen, lodoxamide, loratidine, levocubasstine, mequitazine, oxatomide, phenindamine, phenyltoloxamine, pyrilamine, setastine, tazifylline, temelastine, terfenadine tripelennamine or triprolidine.

Suitable cough suppressants include caramiphen, codeine (codeine phosphate) or dextromethorphan. Suitable decongestants include pseudoephedrine, phenylpropanolamine and phenylephrine. Suitable expectorants include guaiphenesin, potassium citrate, potassium guaiacolsulphonate, potassium sulphate and terpin hydrate. The amounts of these other pharmacologically active ingredients to be used are those known to those skilled in the art. For guidelines as to suitable dosage, reference may be made to MIMS, the Physicians Desk Register and the OTC Handbook.

It is a general component of the present invention that any vasodilator substance can be effectively used as an active ingredient for the reduction of fever in alternative embodiments of the present invention. Examples of such vasodilator substances include but are not limited to: arginine, B3 vitamin derivatives, bencyclane fumarate, benzyl nicotinate, buphenine hydrochloride, ciclonicate, cyclandelate, ethyl nicotinate, hepronicate, hexyl nicotinate, hydralazine, inositol nicotinate, isoxsuprine hydrochloride, methyl nicotinate, minoxidol, naftidrofuryl oxalate, nicametate citrate, niceritrol, nicoboxil, nicofuranose, nicotinyl alcohol, nicotinyl alcohol tartrate, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, vitamin E nicotinate, and xanthinol nicotinate. Centrally acting vasomodulatory agents include clonidine, quanaberz, and methyl dopa. Alpha-adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion-blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH. Angiotensin II analogs include saralasin.

For topical application (e.g., using a gel, ointment, or patch), the discussion above highlighted the preferred use of nitroglycerin. Yet, this example is not intended to be limiting. In particular, the discussion of US 2005/0282870, here incorporated in its entirety, presents several alternative substances and compositions for topical applications which result in skin vasodilation effect which can be used according to the present invention for the treatment of fever condition.

A preferred embodiment which includes a combination of vasodilators with plant extracts includes preparations containing a combination of capsicum oleoresin and methyl nicotinate. Capsicum oleoresin is a naturally-occurring product obtained from a member of the capsicum pepper family. The active ingredient is capsaicin which is used as a counter-irritant. Counter-irritants, when applied to the skin, cause redness and heat to be produced. Methyl nicotinate is known as a rubefacient (i.e., it creates a feeling of warmth when rubbed into the skin). In fact, “warming creams” for muscle pain relief (e.g., Reglex) use it. Similarly, other substances known in the art as rubefecient, may be incorporated as active ingredients in preferred embodiments.

In a further aspect the present invention provides the use of a vasodilator substance in the preparation of a pharmaceutical composition for alleviating fever in a human subject suffering from a fever condition. Preferably, said vasodilator substance is vitamin B3 or a nitric oxide donor, such as nitroglycerine.

Effective dosage of vitamin B3 is between 0.1 mg to 2 mg per body weight of said subject.

The use of vitamin B3 as a vasodilator substance for lowering fever is particularly convenient for the treatment of human newborns, infants, toddlers or children in general.

The use of nitric oxide donors for lowering fever is particularly convenient for the treatment of human adults.

Said composition may further comprise an anti-pyretic substance, wherein said anti-pyretic substance is selected from the group consisting of acetaminophen, acetylsalicylic acid, a non-steroidal anti-inflammatory agent such as ibuprofen, and derivatives thereof.

In another embodiment, the composition is combined with an anti-pyretic substance or a composition comprising the same, wherein said anti-pyretic substance or composition comprising the same is to be administered to said subject before, after or together with said composition comprising said vasodilator substance. Preferably, said anti-pyretic substance is selected from the group consisting of acetaminophen, acetylsalicylic acid, a non-steroidal anti-inflammatory agent such as ibuprofen, and derivatives thereof.

Administration of said composition is via one of oral, topical, sub-lingual, transdermal, rectal or inhalatory routes.

Preferably said composition is for oral administration and is formulated as one of a pill, a capsule, a trochee, a lozenge, a caplet, a syrup, an emulsion, a spray or a suspension liquid.

Alternatively, said composition is for topical administration and is formulated as one of a powder, an ointment, a cream, a gel, a lotion, a spray, or a transdermal patch.

When for rectal administration, said composition is in suppository form.

For inhalatory administration, said composition may be formulated as a spray, a gas, a vapor, or any other form suitable for inhalation.

In another specific embodiment, said composition is formulated for slow release of the vasodilator substance.

In general, core body temperature is reduced by at least 0.5 degrees Celsius within 40 minutes from time of intake of said composition comprising the vasodilator.

In yet another embodiment, said composition comprising the vasodilator further comprises a diaphoretic plant extract substance.

In an even additional embodiment, said composition further comprises another vasodilator substance, said substance being selected from the group consisting of arginine, bencyclane fumarate, benzyl nicotinate, buphenine, histamine, hydrochloride, ciclonicate, cyclandelate, ethyl nicotinate, hepronicate, hexyl nicotinate, hydralazine, inositol nicotinate, isoxsuprine hydrochloride, methyl nicotinate, minoxidol, naftidrofuryl oxalate, nicametate citrate, niceritrol, nicoboxil, nicofuranose, nicotinyl alcohol, nicotinyl alcohol tartrate, nicotinic acid, niacinamide, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, vitamin E nicotinate, and xanthinol nicotinate; centrally acting vasomodulatory agents including clonidine, quanaberz, and methyl dopa; Alpha-adrenoceptor blocking agents include indoramin, phenoxybenzamine, phentolamine, and prazosin. Adrenergic neuron blocking agents include bedmidine, debrisoquine, and guanethidine; ACE inhibitors including benazepril, captopril, cilazapril, enalapril, fosinopril, lisinopril, perindopril, quinapril, and ramipril; ganglion-blocking agents include pentolinium and trimetaphan; calcium channel blockers including amlodipine, diltiazem, felodipine, isradipine, nicardipine, nifedipine, nimodipine; and verapamil; Prostaglandins including prostacyclin, thrombuxane A2, leukotrienes, PGA, PGA1, PGA2, PGE1, PGE2, PGD, PGG, and PGH; Angiotensin II analogs including saralasin.

Alternatively, said other vasodilator substance is selected from the group consisting of Pentoxifylline, Cilostazol, Tolazoline, Phentolamine, Nicergoline, Phenoxybenzamine, and Ergoloid mesylate.

A clear advantage of the method of alleviating fever of the present invention, particularly the combination method, as well as the pharmaceutical composition provided by the present invention is that there is a very quick effect for immediate fever relieve, provided by the action of the vasodilator, which is then prolonged by the action of the anti-pyretic used in combination.

A further aspect of the present invention provides a commercial package for alleviating fever, wherein said package comprises:

(a) a composition comprising a vasodilator substance or a composition comprising the same;

(b) written material containing instructions for use and dosage of the therapeutic contents comprised in the commercial package.

Preferably, said vasodilator substance comprised in the commercial package is vitamin B3 or a NO-donor, such as nitroglycerin.

In one embodiment the commercial package may further comprise an anti-pyretic substance, or a composition comprising the same, said substance being selected from the group consisting of acetaminophen, aspirin, ibuprofen, and derivatives thereof.

In another embodiment the commercial package may additionally comprise a diaphoretic plant extract substance or a composition comprising the same.

In a further embodiment the commercial package further comprises an additional vasodilator substance, said substance being selected from the group consisting of arginine, bencyclane fumarate, benzyl nicotinate, buphenine, histamine, hydrochloride, ciclonicate, cyclandelate, ethyl nicotinate, hepronicate, hexyl nicotinate, hydralazine, inositol nicotinate, isoxsuprine hydrochloride, methyl nicotinate, minoxidol, naftidrofuryl oxalate, nicametate citrate, niceritrol, nicoboxil, nicofuranose, nicotinyl alcohol, nicotinyl alcohol tartrate, nicotinic acid, niacinamide, nitric oxide, nitroglycerin, nonivamide, oxpentifylline, papaverine, papaveroline, pentifylline, peroxynitrite, sodium nitroprusside, suloctidil, teasuprine, thymoxamine hydrochloride, tolazoline, vitamin E nicotinate, and xanthinol nicotinate; centrally acting vasomodulatory agents including clonidine, quanaberz, and methyl dopa; Alpha-adrenoceptor blocking agents include indoramin, phenoxybenzamine, phentolamine, and prazosin. Adrenergic neuron blocking agents include bedmidine, debrisoquine, and guanethidine; ACE inhibitors including benazepril, captopril, cilazapril, enalapril, fosinopril, lisinopril, perindopril, quinapril, and ramipril; ganglion-blocking agents include pentolinium and trimetaphan; calcium channel blockers including amlodipine, diltiazem, felodipine, isradipine, nicardipine, nifedipine, nimodipine, and verapamil; Prostaglandins including prostacyclin, thrombuxane A2, leukotrienes, PGA, PGA1, PGA2, PGE1, PGE2, PGD, PGG, and PGH; Angiotensin II analogs including saralasin.

In one specific embodiment of the commercial package, said composition comprising said vasodilator substance is formulated as one of a pill, a capsule, a trochee, a lozenge, a caplet, a syrup, an emulsion, a suspension liquid, a powder, an ointment, a cream, a gel, a lotion, a transdermal patch, a spray, a gas, a vapor, or a suppository.

As referred to herein, a commercial package may also be referred to as a kit.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the general combination of parts that perform the same functions as exemplified in the embodiments, and includes both combinations and sub-combinations of the various features described hereinabove as well as variations and modifications thereof, which would occur to persons skilled in the art upon reading the foregoing description.

As used in the specification and the appended claims and in accordance with long-standing patent Law practice, the singular forms “a” “an” and “the” generally mean “at least one”, “one or more”, and other plural references unless the context clearly dictates otherwise. Thus, for example “an anti-pyretic substance” and “a vasodilator” include mixture of anti-pyretics or vasodilators of the type described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Clinical Trial

The effect of B3 vitamin (Niacin) intake on core body temperature, alone or in combination with Acetaminophen (combination substance) is tested.

### Dosage Levels

The adult RDA for Vitamin B3 is 20 mg (i.e., about 0.3 mg per Kg weight).

The effect of three dosages is tested:
High (1.5 mg/Kg),
Medium (1 mg/kg),
Low (0.5 mg/Kg).

### Temperature Measurement

From the start of each subject examination (t₀=0) until the end of examination after 6 hours (t=6 h), the subject's core body temperature is recorded every 15 minutes.

Start Combination Substance Administration (t₁)

At t=15 min, the administration of either a conventional dosage of Acetaminophen, typically 15 mg per kg body weight or a placebo is administered.

Start B3 Vitamin Administration (t₂)

The effect of B3 vitamin administration at three different reference times in combination with Acetaminophen is verified:
Simultaneously with Acetaminophen administration (at t₂=15 min),
1.5 h (at t₂=1 h 45 min), which is approximately the time of peak effectiveness of Acetaminophen,
4 h after Acetaminophen (at t₂=4 h 15 min), which is approximately the time of termination of Acetaminophen effectiveness.

### Reference/Placebo

The reference test is the administration of conventional dosage of Acetaminophen+a placebo for B3 vitamin.

Thus, altogether there are 10 different subject groups associated with the various treatment combinations of 3 dosages and 3 administration times of B3 vitamin, and the B3 placebo.

1. A method of reducing fever in a human subject, said method comprising administering to a subject having a fever of at least 38 degrees Celsius an amount of a niacin substance selected from the group consisting of niacin, a pharmaceutically acceptable salt of niacin, an ester of niacin, a pharmaceutically acceptable salt of an ester of niacin, an amide of niacin, a pharmaceutically acceptable salt of an amide of niacin and mixtures thereof, effective to reduce the core body temperature of said subject by at least 0.5° C. within 50 minutes of administration.

2. The method of claim 1, wherein said subject is a child, a toddler or an infant.

3. The method of claim 1, wherein said niacin substance is administered in combination with a nitric oxide donor.

4. The method of claim 3, wherein said subject is an adult.

5. The method claim 1, wherein the administration of said niacin substance is via one of oral, topical, sub-lingual, transdermal, rectal and inhalatory administration.

6. The method of claim 5, wherein said administration is oral administration via a pill, a capsule, a trochee, a lozenge, a caplet, a syrup, an emulsion, a suspension liquid, or a powder.

7. The method of claim 5, wherein said administration is topical administration via an ointment, a cream, a gel, a lotion or a powder.

8. The method of claim 5, wherein said administration is through a transdermal patch.

9. The method of claim 5, wherein said administration is through a spray.

10. The method of claim 1, wherein the administration of said niacin substance is via a composition which is formulated for slow or delayed release.

11. The method of claim 1, wherein the effective dosage of said niacin substance is between 0.1 mg to 2 mg per kg body weight of said subject per hour.

12. The method of claim 1, further comprising administering to said subject a diaphoretic plant extract substance.

13. The method according to claim 12, wherein said niacin substance is administered in a composition which also contains said diaphoretic plant extract substance.

14. The method of claim 1, further comprising administering to said subject an additional substance selected from the group consisting of arginine, bencyclane fumarate, buphenine, histamine, hydrochloride, ciclonicate, cyclandel ate, hepronicate, hydralazine, isoxsuprine hydrochloride, minoxidol, naftidrofuryl oxalate, niceritrol, nicoboxil, nicofuranose, nicotinyl alcohol, nicotinyl alcohol tartrate, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, clonidine, quanaberz, methyl dopa, indoramin, phenoxybenzamine, phentolamine, prazosin, bedmidine, debrisoquine, guanethidine, benazepril, captopril, cilazapril, enalapril, fosinopril, lisinopril, perindopril, quinapril, ramipril, pentolinium, trimetaphan, amlodipine, diltiazem, felodipine, isradipine, nicardipine, nifedipine, nimodipine, verapamil, prostacyclin, thrombuxane A2, leukotrienes, PGA, PGA1, PGA2, PGE1, PGE2, PGD, PGG, PGH, and saralasin.

15. The method according to claim 14, wherein said niacin substance is administered in a composition which also contains said additional substance.

16. The method of claim 1, further comprising administering a further substance selected from the group consisting of Pentoxifylline, Cilostazol, Tolazoline, Phentolamine, Nicergoline, Phenoxybenzamine, and Ergoloid mesylate.

17. The method according to claim 16, wherein said niacin substance is administered in a composition which also contains said further substance.

18. A method according to any preceding claim, wherein said niacin substance is administered in combination with a COX-2 inhibiting substance, wherein said COX-2 inhibiting substance is administered before, after or together with said niacin substance.

19. The method of claim 18, wherein said COX-2 inhibiting substance is selected from the group consisting of acetaminophen, acetylsalicylic acid, a non-steroidal anti-inflammatory agent other than aspirin, and derivatives thereof.

20. The method according to claim 1, wherein said niacin substance is administered in a composition which is substantially free of other therapeutic agents.

21. The method according to claim 1 or claim 20, wherein said niacin substance is administered as a monotherapy.

22. A commercial package, said package comprising:
(a) a niacin substance selected from the group consisting of niacin, a pharmaceutically acceptable salt of niacin, an ester of niacin, a pharmaceutically acceptable salt of an ester of niacin, an amide of niacin, a pharmaceutically acceptable salt of an amide of niacin and mixtures thereof; and
(b) written material containing instructions for use and dosage of said niacin substance for reducing fever.

23. The commercial package of claim 22, further comprising a COX-2 inhibiting substance or a composition comprising the same.

24. The commercial package of claim 23, wherein said COX-2 inhibiting substance is selected from the group consisting of acetaminophen, aspirin, ibuprofen, and derivatives thereof.

25. The commercial package of claim 22, further comprising a diaphoretic plant extract substance.

26. The commercial package of claim 23, further comprising an additional substance selected from the group consisting of arginine, bencyclane fumarate, buphenine, histamine, hydrochloride, ciclonicate, cyclandelate, hepronicate, hydralazine, isoxsuprine hydrochloride, minoxidol, naftidrofuryl oxalate, niceritrol, nicoboxil, nicofuranose, nicotinyl alcohol, nicotinyl alcohol tartrate, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, clonidine, quanaberz, methyl dopa, indoramin, phenoxybenzamine, phentolamine, prazosin, bedmidine, debrisoquine, guanethidine, benazepril, captopril, cilazapril, enalapril, fosinopril, lisinopril, perindopril, quinapril, ramipril, pentolinium, trimetaphan, amlodipine, diltiazem, felodipine, isradipine, nicardipine, nifedipine, nimodipine, verapamil, prostacyclin, thrombuxane A2, leukotrienes, PGA, PGA1, PGA2, PGE1, PGE2, PGD, PGG, PGH, and saralasin.

27. The commercial package of claim 22, further comprising a nitric oxide donor.

28. The commercial package of claim 22, wherein said niacin substance is present in a composition which is in a form selected from a pill, a capsule, a trochee, a lozenge, a caplet, a syrup, an emulsion, a suspension liquid, a powder, an ointment, a cream, a gel, a lotion, a spray, and a transdermal patch.

29. The package of claim 22, which is substantially free of other therapeutic agents.

30. A pharmaceutical composition comprising (a) a niacin substance selected from the group consisting of niacin, an ester of niacin, a pharmaceutically acceptable salt of niacin, an amide of niacin, a pharmaceutically acceptable salt of an amide of niacin, and mixtures thereof, and (b) acetaminophen.

31. The composition of claim 30, wherein the composition is formulated for oral, sub-lingual, rectal or inhalatory administration.

32. The pharmaceutical composition of claim 30, wherein the weight:weight ratio of said niacin substance to acetaminophen is between 1:30 and 1:3.

33. The pharmaceutical composition of claim 30, wherein said composition is formulated as a pill, a capsule, a trochee, a lozenge, a caplet, a syrup, an emulsion, a suspension liquid, a powder, a spray or a suppository.

34. The pharmaceutical composition of claim 33, further comprising a pharmaceutically acceptable adjuvant, carrier, excipient or diluent.

35. The pharmaceutical composition of claim 30, wherein the amount of said niacin substance is sufficient to reduce core body temperature of a human subject having a fever of at least 38 degrees Celsius which ingests said composition by at least 0.5 degrees Celsius within 50 minutes from time of administration of said composition.

36. The pharmaceutical composition of claim 30, further comprising a diaphoretic plant extract substance.

37. The pharmaceutical composition of any of claims 30 to 36, further comprising an additional therapeutic compound, said additional therapeutic compound being selected from the group consisting of an anti-histamine, a cough suppressant, a decongestant, an expectorant, a muscle-relaxant, an analgesic, caffeine, an antibiotic, an anti-inflammatory, and mixtures thereof.

38. The pharmaceutical composition of any of claims 30 to 36, further comprising a nitric oxide donor.

39. Use of a niacin substance selected from the group consisting of niacin, a pharmaceutically acceptable salt of niacin, an ester of niacin, a pharmaceutically acceptable salt of an ester of niacin, a pharmaceutically acceptable salt of niacin, an amide of niacin, a pharmaceutically acceptable salt of an amide of niacin, and mixtures thereof in the preparation of a pharmaceutical composition for reducing a fever of at least 38° C. in a human subject by at least 0.5° C. within 50 minutes of administration.

40. The use of claim 39, wherein said human subject is an infant, a toddler or a child.

41. The use of claim 39, wherein said composition further comprises a nitric oxide donor.

42. The use of claim 39, wherein said human subject is an adult.

43. The use of claim 39, wherein said composition is administrable via an oral, topical, sub-lingual, rectal, inhalatory or transdermal route.

44. The use of any of claim 43, wherein said composition is for oral administration and is formulated as one of a pill, a capsule, a trochee, a lozenge, a caplet, a syrup, an emulsion or a suspension liquid.

45. The use of claim 39, wherein said composition is for topical administration and is formulated as one of a powder, an ointment, a cream, a gel, or a lotion.

46. The use of claim 39, wherein said composition is for topical administration and is formulated as a transdermal patch.

47. The use of claim 39, wherein said composition is for inhalatory administration and is formulated as a spray.

48. The use of claim 39, wherein said composition is a slow or delayed release composition.

49. The use of claim 39, wherein the effective dose of said niacin substance is from 0.1 mg to 2 mg per kg body weight of said subject per hour.

50. The use of claim 39, wherein said composition further comprises a diaphoretic plant extract substance.

51. The use of any of claims 39- to 51, wherein the effective dosage of said niacin substance is between 0.2 mg to 2 mg per kg body weight of said subject.

52. The use of any claim 39, wherein said composition further comprises an additional substance selected from the group consisting of arginine, bencyclane fumarate, buphenine, histamine, hydrochloride, ciclonicate, cyclandelate, hepronicate, hydralazine, isoxsuprine hydrochloride, minoxidol, naftidrofuryl oxalate, niceritrol, nicoboxil, nicofuranose, nicotinyl alcohol, nicotinyl alcohol tartrate, nitric oxide, nitroglycerin, nonivamide, oxpentifylline, papaverine, papaveroline, pentifylline, peroxynitrite, pinacidil, sodium nitroprusside, suloctidil, teasuprine, thymoxamine hydrochloride, tolazoline, clonidine, quanaberz, methyl dopa, indoramin, phenoxybenzamine, phentolamine, prazosin, bedmidine, debrisoquine, guanethidine, benazepril, captopril, cilazapril, enalapril, fosinopril, lisinopril, perindopril, quinapril, ramipril, pentolinium, trimetaphan, amlodipine, diltiazem, felodipine, isradipine, nicardipine, nifedipine, nimodipine, verapamil, prostacyclin, thrombuxane A2, leukotrienes, PGA, PGA1; PGA2, PGE1, PGE2, PGD, PGG, PGH, and saralasin.

53. The use of claim 39, wherein said composition comprises a further substance selected from the group consisting of Pentoxifylline, Cilostazol, Tolazoline, Phentolamine, Nicergoline, Phenoxybenzamine, and Ergoloid mesylate.

54. The use of any one of claims 39 to 53, wherein said composition further comprises a COX-2 inhibiting substance which is selected from the group consisting of acetaminophen, aspirin, a non-steroidal anti-inflammatory other than aspirin, and derivatives thereof.

55. The use of claim 54, wherein said COX-2 inhibiting substance is selected from the group consisting of acetaminophen, acetylsalicylic acid, a non-steroidal anti-inflammatory agent other than aspirin, and derivatives thereof.

56. The use of claim 39, wherein said composition is substantially free of other therapeutic agents.

57. The uses of claim 39 or claim 56, wherein said composition is intended for administration as a monotherapy.

58. A method according to claim 1, said amount of said niacin substance is effective to reduce the core body temperature of said subject by at least 0.5° C. within 40 minutes of administration.

59. The pharmaceutical composition of claim 35, wherein said amount of said niacin substance is sufficient to reduce core body temperature of said human subject by at least 0.5 degrees Celsius within 40 minutes from administration of said composition.

60. Use of a niacin substance according to claim 39, wherein said composition is for reducing a fever of at least 38° C. in a human subject by at least 0.5° C. within 40 minutes of administration.

61. A method of reducing fever in a human subject, said method comprising administering to a subject having a fever of at least 38 degrees Celsius a delayed release composition comprising an amount of a niacin substance selected from the group consisting of niacin, a pharmaceutically acceptable salt of niacin, an ester of niacin, a pharmaceutically acceptable salt of an ester of niacin, an amide of niacin, a pharmaceutically acceptable salt of an amide of niacin and mixtures thereof, effective to reduce the core body temperature of said subject by at least 0.5° C. within 50 minutes of delayed release.
